(19) United States
(12) Plant Patent Application Publication
(10) Pub. No.: US 2003/0051284 P1
(43) Pub. Date: Mar. 13, 2003

(54) Euphorbia interspecific hybrid plant named "Eckcory"

(76) Inventor: Ruth S. Kobayashi, (US)

Correspondence Address: Robert E. Hanson, FULBRIGHT & JAWORSKI, L.L.P., Ste. 2400, 600 Congress Ave, Austin, TX, 78701, US

(21) Appl. No.: 09/947,586

(22) Filed: Sep. 4, 2001

(51) Int. Cl. A01H 5/00

(52) U.S. Cl. PLT/302

(57) ABSTRACT

The variety “Eckcory” is a new and distinct interspecific hybrid plant. The plant is characterized by clean and dark green foliage, eliptical leaves that have acute bases and acuminate tips, brightly colored pink and white brachts and strong lateral branching.

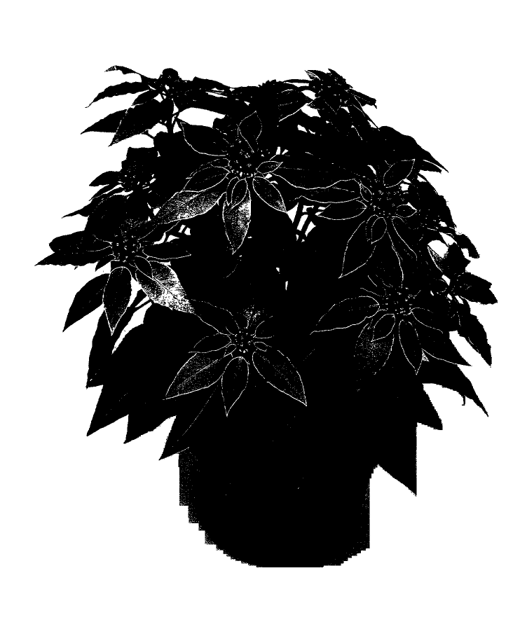

### CROSS-REFERENCE TO RELATED APPLICATIONS

This application claims the priority of U.S. patent application Ser. No. 09/690,370, filed Oct. 16, 2000.

### BACKGROUND OF THE INVENTION

The invention relates generally to the new and distinct variety of interspecific plant termed “Eckcory.” The new plant was created in my greenhouse and laboratory in Encinitas, Calif. The plant was prepared by crossing the Euphorbia pulcherrima variety “M-6,” a Paul Ecke Ranch proprietary variety, with a seedling of the uncultivated species Euphorbia cornastra. The variety was selected by excision of a newly formed embryo from a seedpod followed by growth into a plantlet on nutrient agar medium. The cultured plantlet was transplanted to a greenhouse growing-medium, where it developed into the mature plant of the invention. As the plant matured, it exhibited strong terminal dominance with little lateral branch development. To induce lateral branching, the new plant was grafted onto a “selfbranching” rootstock by application of the procedures set forth in U.S. Pat. No. 4,724,276.

The plant of the invention is distinct from the parent plants from which it was derived. For example, Euphorbia pulcherrima is a “short day” plant with respect to flower initiation, while Euphorbia cornastra is a “long day” plant. However, the interspecific hybrid flowered in response to short days and the time to full flower development was found to be a relatively short six weeks, compared to the E. pulcherrima parent that took nine weeks for flower development under the same growing environment.

The first vegetative propagation of the plant in vitro occurred in October, 1998 in Encinitas Calif. The plant was first propagated ex vitro by grafting in August, 1999 in Encinitas, Calif. The distinctive characteristics of the plant remained consistent following propagation.

### BRIEF DESCRIPTION OF THE DRAWING

The plant of the invention is illustrated in the accompanying color photographs.

### DETAILED DESCRIPTION OF THE VARIETY

The following is a detailed description of the new plant as observed in Encinitas, Calif., USA during May 2000. Observations were recorded from flowering plants, grown as one branched plant per pot. The pot was 17 cm in diameter and 13 cm in height. Color designations are compared to the 1986 edition of RHS Colour Chart, first published in 1966 by The Royal Horticultural Society, London, England.

| | |
|---|---|
| Origin: | An interspecific hybrid created by cross-pollinating a |
| | cultivated Euphorbia pulcherrima plant with an |
| | uncultivated Euphorbia cornastra plant. |
| Classification: | Botanical - Euphorbia X hybrid |
| Form: | Shrub |
| Height: | Short-medium |
| Growth | Flowering was observed, branched plant in a pot with an |
| Habit: | overall height of 42 cm, including the pot and an overall |
| | width of 42 cm. The plant grew upright but was |
| | somewhat spreading. The average bract diameter of |
| | individual flowers was 20 cm. |
| Branching: | Lateral branches develop and terminate in a flower |
| | without pinching. However, when pinched before flower |
| | induction to remove terminal dominance, all lateral |
| | branches develop uniformly and at a faster rate. The plant |
| | of description was pinched leaving 12 nodes on the stem. |
| | Strong lateral branches of nearly equal length and vigor |
| | arose, one from each stem node, resulting in a plant with |
| | 12 lateral branches. The lowermost lateral had 14 nodes |
| | and the uppermost, 9 nodes subtending the flower. |
| Growth Rate: | Rooting of stem cutting occurs in 18-21 days under |
| | intermittent mist. The plant will flower in about 6 weeks |
| | under continuous long night conditions and night |
| | temperatures of about 16-18 degrees C. |
| Foliage: | The foliage is clean and dark green from bottom to top of |
| | the plant. The leaves are of medium size, leaf blades |
| | typically 10 cm long and 5 cm wide for the larger lower |
| | leaves and 6 cm long and 2.5 cm wide for the smaller |
| | upper leaves. Leaf petioles are green and 6 cm long on |
| | the larger leaves to 3 cm on the smaller leaves. The upper |
| | leaf surface is glabrous, but slightly rugose and the under |
| | surface is finely pubescent. |
| Leaf shape: | Leaves are elliptic with acute bases and acuminate tips. |
| | Leaf margins are entire. |
| Color: | Upper side—Dark green, darker than RHS 139A |
| | Under side—Green, near RHS 147B |
| Bracts: | There were 2-4 pink transitional bracts at the upper |
| | stem nodes just below the inflorescence. These were 7-8 |
| | cm long and 3 cm wide. Eight to 10 bracts subtending the |
| | cyathia varied in size from 5 cm long and 2 cm wide to |
| | 1.5 cm long and 1 cm wide. |
| Shape: | Bracts are elliptic with acute bases and acuminate tips. |
| | Bract margins are entire. |
| Color: | Upper side—Pink, near RHS 57D |
| | Under side—Grayish-White, near RHS 156D |
| Flowers: | Generally, 8-10 rudimentary and sterile cyathia (flowers) |
| | were present at maturity. Each cyathium was about 5 mm |
| | long and 3 mm wide and green in color. |

What is claimed is:

1. The new and distinct interspecific hybrid plant variety “Eckcory” as illustrated and described herein.
